(19) United States
(12) Patent Application Publication
(10) Pub. No.: US 2026/0306203 A1
(43) Pub. Date: Oct. 8, 2026

(54) SURGICAL ROBOTIC SYSTEM AND METHOD FOR DIGITAL TWIN GENERATION

(71) Applicant: Covidien LP, Mansfield, MA (US)

(72) Inventors: David J. Hirvonen, Brooklyn, NY (US); Max Loeb Balter, Boston, MA (US); Michael A. Eiden, Somerville, MA (US); Matthew Steven Euliano, Boston, MA (US); Tuvia Chaim Rappaport, Somerville, MA (US); Emily Marie Ludwig, New Haven, CT (US); Donald E. Barry, Jr., Groton, MA (US); Richard O. Kuenzler, Framingham, MA (US); Faisal I. Bashir, Andover, MA (US); Steven J. Levine, Franklin, MA (US); William J. Peine, Ashland, MA (US)

(21) Appl. No.: 19/483,137

(22) PCT Filed: May 16, 2024
(86) PCT No.: PCT/US2024/029586

§ 371 Date: Nov. 10, 2025

Related U.S. Application Data
(60) Provisional application No. 63/467,038, filed on May 17, 2023.

(51) Int. Cl. A61B 34/37; A61B 34/00; A61B 34/10; A61B 34/20; A61B 34/30

(52) U.S. Cl. CPC A61B 34/37; A61B 34/10; A61B 34/20; A61B 34/25; A61B 2034/102; A61B 2034/2055; A61B 2034/301

(57) ABSTRACT

A surgical robotic system includes a plurality of robotic arms each of which includes a surgical instrument, and a surgeon console configured to receive input to control a robotic arm of the plurality of robotic arms. The system also includes a controller configured to generate a 3D virtual model that may include a plurality of virtual arms replicating the plurality of robotic arms, where the 3D virtual model is moved in response to the input, and a display screen configured to display the 3D virtual model.

### CROSS-REFERENCE TO RELATED APPLICATION

The present application claims priority to and benefit of U.S. Provisional Patent Application No. 63/467,038, filed on May 17, 2023, the entire disclosure of which is incorporated by reference herein.

### BACKGROUND

Surgical robotic systems are currently being used in a variety of medical procedures, including minimally invasive surgical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. A variety of different types of instruments are used with surgical robotic systems that are designed to perform specific functions, such as steerable catheters and endoscopes. During use, surgical robotic systems generate lots of data from multiple sensors and other devices, which are used to monitor and control the surgical robotic systems.

### SUMMARY

According to one aspect of the present disclosure, a surgical robotic system is disclosed. The surgical robotic system includes a plurality of robotic arms each of which includes a surgical instrument, and a surgeon console configured to receive input to control a robotic arm of the plurality of robotic arms. The system also includes a controller configured to generate a 3D virtual model which may include a plurality of virtual arms replicating the plurality of robotic arms, where the 3D virtual model is moved in response to the input, and a display screen configured to display the 3D virtual model.

Implementations of the above embodiment may include one or more of the following features. According to one aspect of the above embodiment, the controller may be further configured to detect a collision between at least two robotic arms of the plurality of robotic arms. The controller may be further configured to predict a collision between at least two robotic arms of the plurality of robotic arms. The controller may also be configured to generate a range of motion limits for the surgical instrument. The display screen may be further configured to display the range of motion limits. The controller may be additionally configured to deregister a virtual arm of the 3D virtual model from a corresponding robotic arm of the plurality of robotic arms. The surgeon console may be configured to control a virtual arm of the plurality of virtual arms in response to the input without controlling the robotic arm of the plurality of robotic arms. The surgeon console may be configured to control one or more virtual arms in response to the input and the robotic arm of the plurality of robotic arms. The surgical robotic system may include a secondary control console configured to receive input to control the virtual arm of the plurality of virtual arms.

According to another embodiment of the present disclosure, a surgical robotic system is disclosed. The surgical robotic system may include a surgeon console for input reception. The system may include a control tower connected to a plurality of movable carts, each cart supporting a robotic arm equipped with a surgical instrument or an imaging device, where the control tower processes input from the surgeon console to control movements of the robotic arms and attached instruments or imaging device. The system may include a video processing device configured to output a processed video stream of a surgical site. The system may include a digital twin generator configured to create a real-time virtual representation of the robotic arms and instruments, displayed on a surgeon console display, enhancing operational control and surgical precision.

Implementations of the above embodiment may include one or more of the following features. According to one aspect of the above embodiment, the digital twin generator utilizes sensor data from the robotic arms to update the virtual representation in real-time. The surgeon console display provides a multi-view display of the surgical site and the digital twin. The digital twin simulates potential movements of the robotic arms and instruments. The system further may include an adaptive movement control mechanism for the robotic arms to avoid detected potential collisions.

According to a further embodiment of the present disclosure, a method for operating a surgical robotic system. The method includes receiving input at a surgeon console. The method also includes controlling a plurality of robotic arms based on the received input, each robotic arm equipped with a surgical instrument or an imaging device. The method further includes generating a 3D virtual model that replicates the plurality of robotic arms and their movements in real-time. The method additionally includes displaying the 3D virtual model on a display screen to provide a visual aid to the surgeon.

Implementations of the above embodiment may include one or more of the following features. According to one aspect of the above embodiment, the method may include predicting potential collisions between the robotic arms based on their movements. Generating the 3D virtual model may include simulating interaction forces between instruments and tissue. The method may also include adjusting the movements of the robotic arms to avoid predicted collisions. Displaying the 3D virtual model may include providing visual indications of range of motion limits for the surgical instruments. The method may include using the 3D virtual model to simulate surgical procedures prior to execution.

### BRIEF DESCRIPTION OF THE DRAWINGS

Various embodiments of the present disclosure are described herein with reference to the drawings wherein:

FIG. 1 is a perspective view of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of the surgical robotic system of FIG. 1 positioned about a surgical table according to an embodiment of the present disclosure;

FIG. 6 is a schematic diagram of a system for determining phases of a surgical procedure according to an embodiment of the present disclosure;

FIG. 7 is a schematic diagram illustrating generation of a digital twin of the surgical robotic system of FIG. 1 according to an embodiment of the present disclosure;

FIG. 8 is a perspective view of the surgeon console displaying a 3D model of the digital twin on first and second display screens according to an embodiment of the present disclosure;

FIG. 9 is a screenshot of a graphical user interface (GUI) for adjusting views of the 3D model of the digital twin according to an embodiment of the present disclosure;

FIG. 10 is a perspective view of a handle controller of the surgeon console for adjusting views of the 3D model of the digital twin according to an embodiment of the present disclosure;

FIG. 11 is a screenshot of a laparoscopic video feed extended by a virtual view provided by the digital twin according to an embodiment of the present disclosure;

FIG. 12 is a screenshot of a laparoscopic video feed including an augmented reality (AR) overlay generated by the digital twin illustrating force imparted on the instrument according to an embodiment of the present disclosure;

FIG. 13 is a screenshot of a laparoscopic video feed including an AR overlay generated by the digital twin showing insertion trajectory of the instrument according to an embodiment of the present disclosure; and

FIG. 14 is a flow chart of a method for generating and using a digital twin for the surgical robotic system according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives user input through one or more interface devices. The input is processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands and to generate a torque commands for activating one or more actuators of the robotic arm, which would, in turn, move the robotic arm in response to the movement commands.

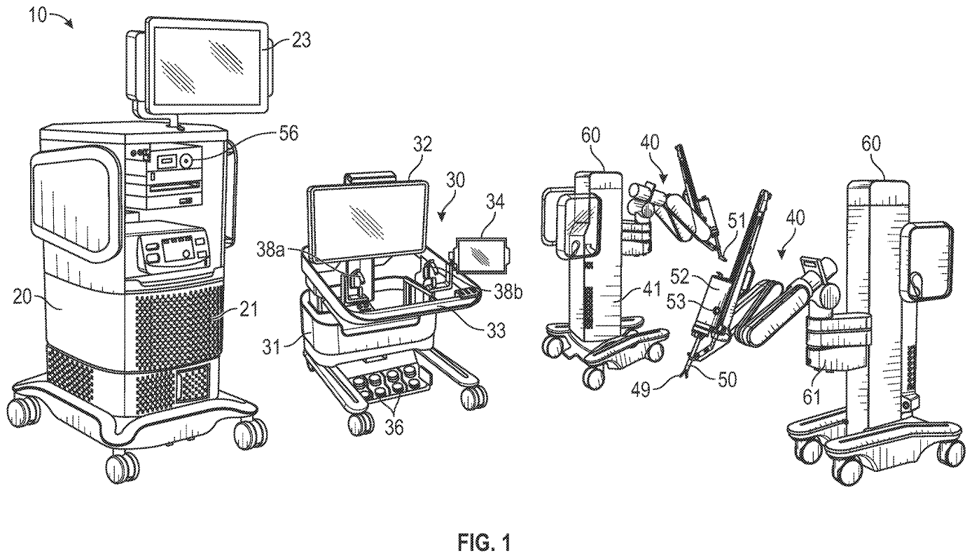

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured to apply a surgical clip onto tissue. However, it will be understood that various types of surgical instruments for use during minimally invasive surgical procedures are contemplated and within the scope of this disclosure.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by camera 51 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen that may display the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the handle controllers 38a and 38b, repositioning camera movement and electrosurgical activation/deactivation. In particular, the foot pedals 36 may be used to perform a clutching action on the handle controllers 38a and 38b. Clutching is initiated by pressing one of the foot pedals 36, which disconnects (i.e., prevents movement inputs) the handle controllers 38a and/or 38b from the robotic arm 40 and corresponding instrument 50 or camera 51 attached thereto. This allows the user to reposition the handle controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted by using any logic processor (e.g., control circuit) adapted to execute algorithms, calculations, and/or set of instructions described herein.

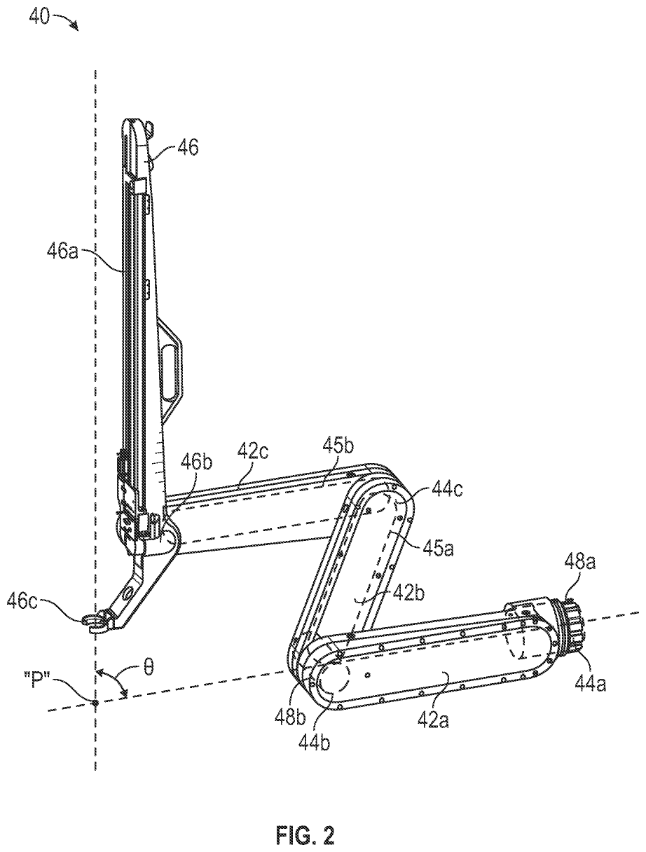

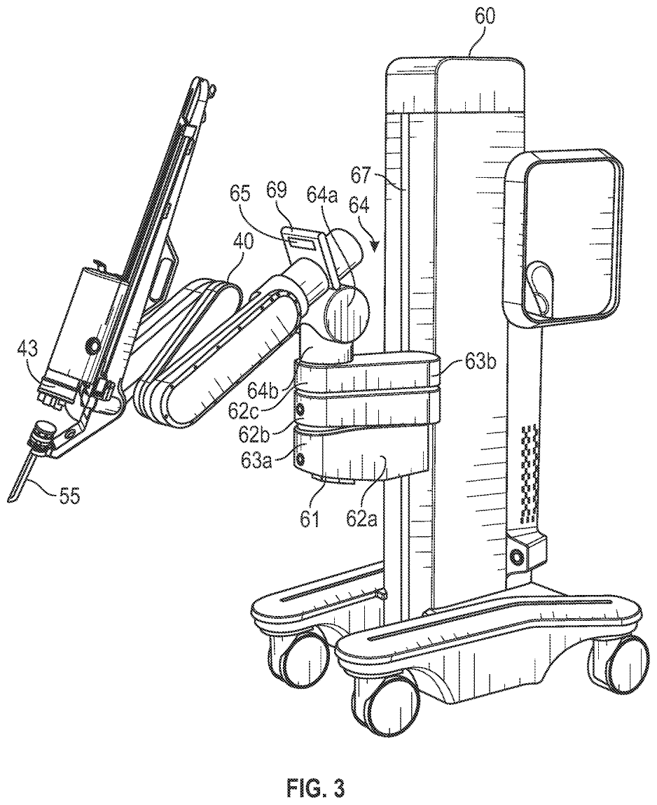

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components of an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52.

The robotic arm 40 also includes a plurality of manual override buttons 53 (FIG. 1) disposed on the IDU 52 and the setup arm 61, which may be used in a manual mode. The user may press one or more of the buttons 53 to move the component associated with the one or more buttons 53.

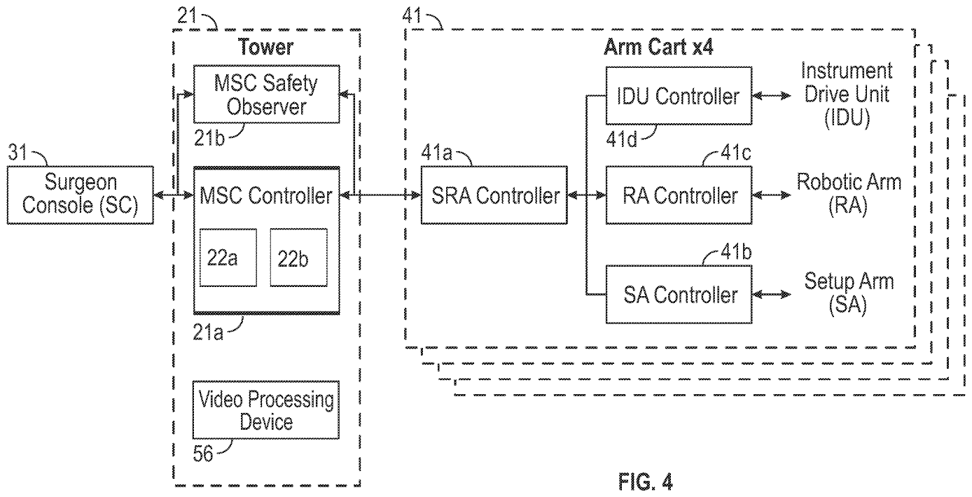

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The controller 21a is coupled to a storage 22a, which may be non-transitory computer-readable medium configured to store any suitable computer data, such as software instructions executable by the controller 21a. The controller 21a also includes transitory memory 22b for loading instructions and other computer readable data during execution of the instructions. In embodiments, other controllers of the system 10 include similar configurations.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The desired angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c. In aspects, handle controller 38a may be substituted for and/or employed in conjunction with handle controller 38b. While reference is made above to handle controller 38a, handle controller 38b may also be used in a similar manner.

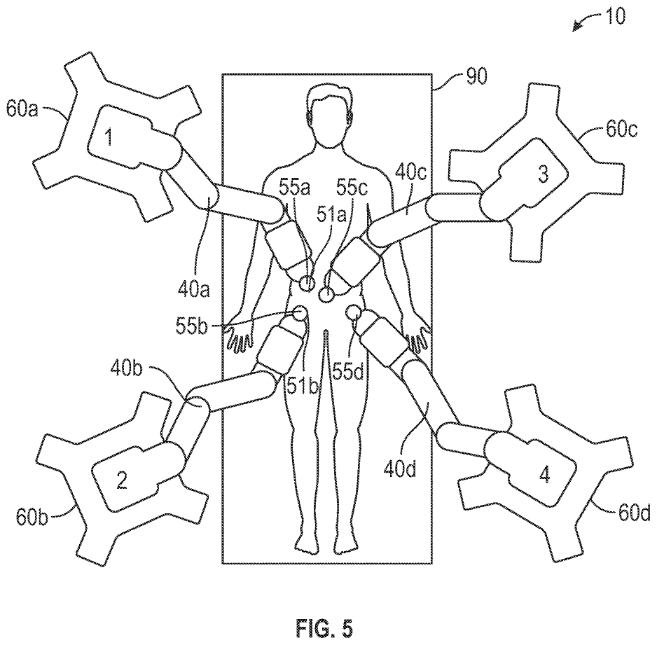

With reference to FIG. 5, the surgical robotic system 10 is set up around a surgical table 90. The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46.

A surgical procedure may include multiple phases, and each phase may include one or more surgical actions. As used herein, the term “phase” represents a surgical event that is composed of a series of steps (e.g., closure). A “surgical action” may include an incision, a compression, a stapling, a clipping, a suturing, a cauterization, a sealing, or any other such actions performed to complete a phase in the surgical procedure. A “step” refers to the completion of a named surgical objective (e.g., hemostasis). During each step, certain surgical instruments 50 (e.g., forceps) are used to achieve a specific objective by performing one or more surgical actions.

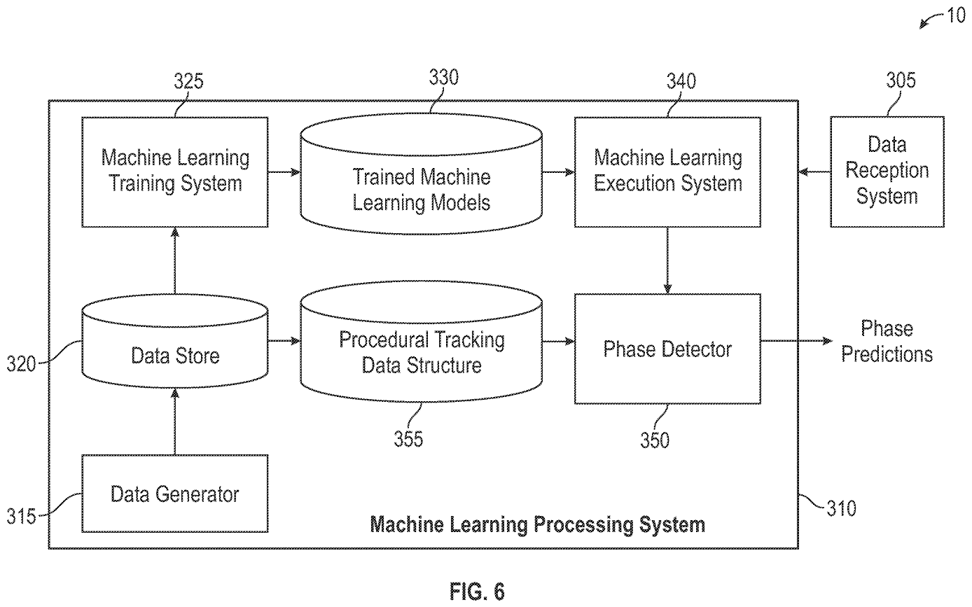

With reference to FIG. 6, the surgical robotic system 10 may include an AI/ML processing system 310 that processes the surgical data using one or more ML models to identify one or more features, such as surgical phase, instrument, anatomical structure, etc., in the surgical data. The ML processing system 310 includes a ML training system 325, which may be a separate device (e.g., server) that stores its output as one or more trained ML models 330. The ML models 330 are accessible by a ML execution system 340. The ML execution system 340 may be separate from the ML training system 325, namely, devices that “train” the models are separate from devices that “infer,” i.e., perform real-time processing of surgical data using the trained ML models 330.

System 10 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed.

The ML processing system 310, in some examples, may further include a data generator 315 to generate simulated surgical data, such as a set of virtual or masked images, or record the video data from the image processing device 56, to train the ML models 330 as well as other sources of data, e.g., user input, arm movement, etc. Data generator 315 can access (read/write) a data store 320 to record data, including multiple images and/or multiple videos.

The ML processing system 310 also includes a phase detector 350 that uses the ML models to identify a phase within the surgical procedure. Phase detector 350 uses a particular procedural tracking data structure 355 from a list of procedural tracking data structures. Phase detector 350 selects the procedural tracking data structure 355 based on the type of surgical procedure that is being performed. In one or more examples, the type of surgical procedure is predetermined or input by user. The procedural tracking data structure 355 identifies a set of potential phases that may correspond to a part of the specific type of surgical procedure.

In some examples, the procedural tracking data structure 355 may be a graph that includes a set of nodes and a set of edges, with each node corresponding to a potential phase. The edges may provide directional connections between nodes that indicate (via the direction) an expected order during which the phases will be encountered throughout an iteration of the surgical procedure. The procedural tracking data structure 355 may include one or more branching nodes that feed to multiple next nodes and/or may include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state may indicate a complication (e.g., blood clots, clogged arteries/veins, etc.), pre-condition (e.g., lesions, polyps, etc.). In some examples, the ML models 330 are trained to detect an “abnormal condition,” such as hemorrhaging, arrhythmias, blood vessel abnormality, etc.

The phase detector 350 outputs the phase prediction associated with a portion of the video data that is analyzed by the ML processing system 310. The phase prediction is associated with the portion of the video data by identifying a start time and an end time of the portion of the video that is analyzed by the ML execution system 340. The phase prediction that is output may include an identity of a surgical phase as detected by the phase detector 350 based on the output of the ML execution system 340. Further, the phase prediction, in one or more examples, may include identities of the structures (e.g., instrument, anatomy, etc.) that are identified by the ML execution system 340 in the portion of the video that is analyzed. The phase prediction may also include a confidence score of the prediction. Other examples may include various other types of information in the phase prediction that is output. The predicted phase may be used by the controller 21a to determine when to enable robotic suturing assistance mode as well as other automatic determinations described above.

The present disclosure provides for generating and using a digital twin for the surgical robotic system 10. The digital twin is a virtual model that mirrors the physical system 10 in real time. The digital twin may be simulated/generated by a digital twin generator embodied as software instructions stored in storage 22a and executed by the controller 21a. The digital twin integrates the physical attributes of the robotic arms 40a-d and their instruments 50 and camera 51, the control mechanisms via the console 30, and the dynamic interactions during surgical procedures. It captures both the hardware aspects, such as the geometric configurations and physical properties of the robotic arms 40a-d and instruments 50, and the software layers, including control algorithms, sensor feedback loops, and user interface dynamics.

The digital twin utilizes data from various sources—sensors on the robotic arms 40a-d, input from the surgeon's console 30, and preoperative surgical plans—to simulate and predict the behavior of the surgical system 10 under different scenarios. The digital twin can model the kinematics and dynamics of each robotic arm 40a-d, the interaction forces between instruments 50 and tissue, and the response to surgeon inputs. This model is continuously updated with real-time data, allowing for a highly accurate reflection of the physical state of system 10 at any moment.

A method 140 for generating and using a digital twin for the surgical robotic system is shown in FIG. 14. At step 142, the method includes receiving input at the surgeon console 30, e.g., through one or more handle controllers 38a and 38b. At step 144, the method also includes controlling the robotic arms 40a-d based on the received input, each robotic arm equipped with a surgical instrument 50 or an imaging device, e.g., camera 51. The method further includes generating a 3D virtual model that replicates the plurality of robotic arms and their movements in real-time at step 146. The method additionally includes displaying the 3D virtual model on a display screen to provide a visual aid to the surgeon at step 148. The steps 146 and 148 are described in further detail below.

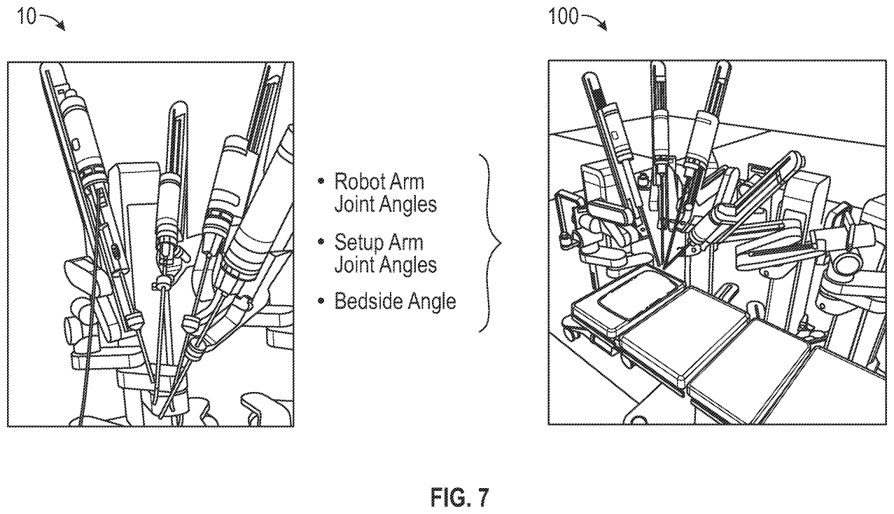

With reference to FIG. 7, a digital twin 100, which is shown as a 3D virtual model, is generated from data provided by the system 10 using data, which includes, robot arm joint angles, setup arm joint angles, bedside angles, and other data from the physical system 10. The digital twin 100 may be generated at the start of the procedure following the set up of the system 10 as shown in FIG. 5. The digital twin 100 may also be generated based on 3D model data of the robotic arms 40a-d and other components as well as any other specification information, e.g., dimensions, functionality, etc. of the robotic system 10. External imaging (e.g., cameras) may also be used to track position of the system 10 to construct the digital twin 100.

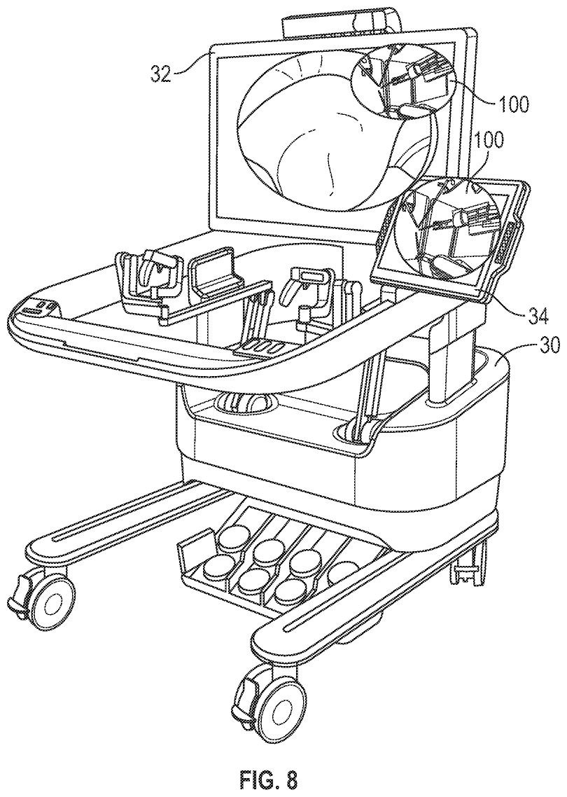

The digital twin 100 may be displayed on one or both of the display screens 32 and 34 of the surgeon console 30 as shown in FIG. 8. Thus, the visualization is provided from any viewpoint of the digital twin 100 either on the stereoscopic first display screen 32 or the side second display screen 34. The digital twin 100 is rendered in 3D, which may be done in high fidelity, accurately representing the physical appearance, colors, and textures of the robotic arms and instruments. This realism aids in understanding the spatial relationships and functionalities of different components. Dynamic lighting and shadow effects may also be used in the digital twin 100 to improve depth perception, making it easier for users to gauge the positions and movements of robotic arms relative to each other and the surgical environment.

The digital twin 100 may be used to provide different viewpoints on the screens 32 and 34. Navigation and/or manipulation of the digital twin 100 allows the surgeon to explore the virtual model from various angles and perspectives, enhancing their understanding and control over the system. The digital twin 100 may include a 3D visualization interface, which provides interactive navigation controls. Suitable controls include orbiting, zooming, panning, preset views, and the like. Users can orbit around the digital twin, viewing it from any horizontal or vertical angle. This may be achieved by clicking and dragging the mouse or using touch gestures on touch-enabled devices. Scrolling or pinch-zoom gestures allow users to zoom in and out, enabling detailed inspection of components or a broader view of the entire system. Horizontal and vertical panning controls move the viewpoint across the model's plane, useful for shifting the focus without altering the viewing angle. Preset perspective view provides a natural viewpoint, mimicking human vision, ideal for understanding the 3D spatial arrangements and depth relationships between components.

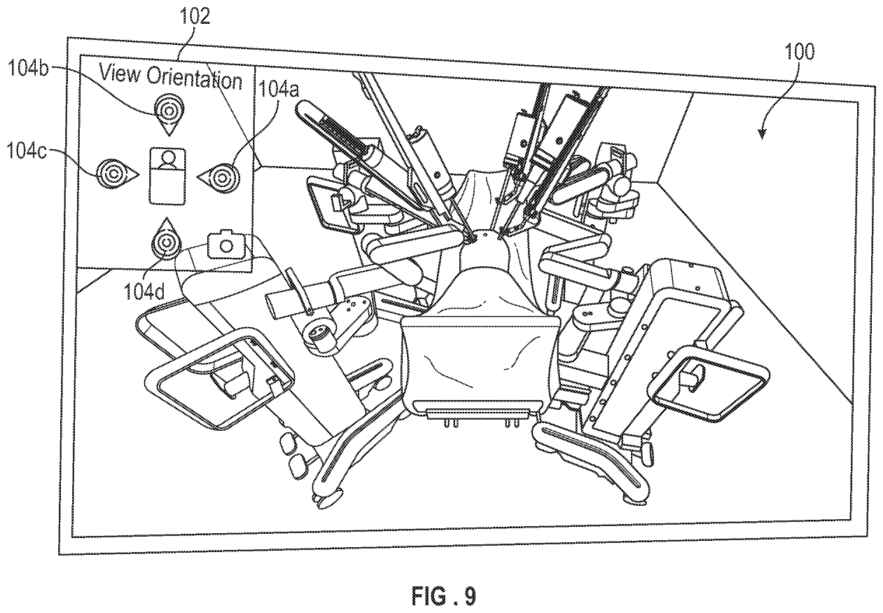

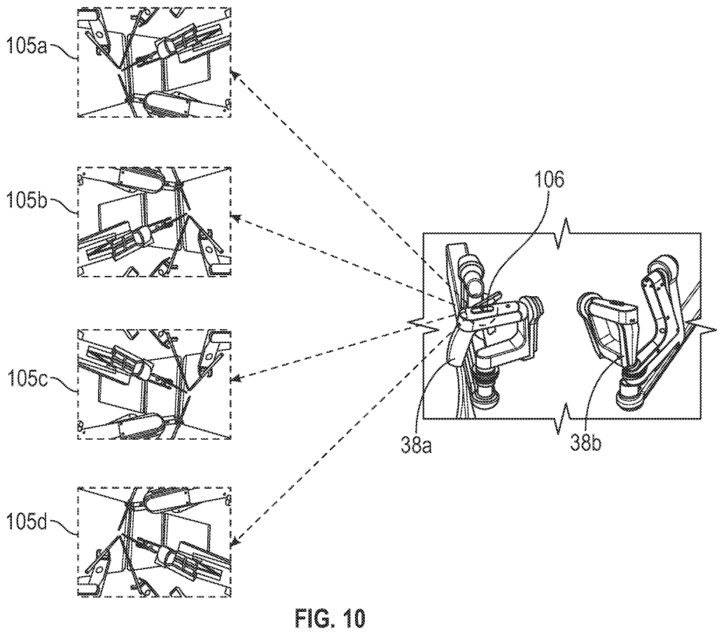

With reference to FIG. 9, the navigation controls may be implemented as touchscreen gestures in a GUI 102 shown on the second display screen 32. The GUI 102 may include a plurality of preset buttons 104a-d, which may be used to select a corresponding views 105a-d (FIG. 10), respectively. In further embodiments, different preset views 105a-d may be selected using buttons 106 on the handle controllers 38a and 38b as shown in FIG. 10. The handle controllers 38a and 38b may be then used to finetune the view by moving the handle controller, e.g., orbiting, zooming, panning, etc.

In addition to viewing controls, the digital twin 100 may also provide interactive features allowing for selection of components, e.g., virtual robotic arms and instruments, by clicking or tapping on components to display detailed information, such as specifications, operational status, or real-time data from sensors. The digital twin 100 may also provide simulation controls, which allow the users to initiate and control simulations of surgical procedures, observing the movements of robotic arms and instruments in response to input from the surgeon's console 30.

To predict and detect potential collisions within a digital twin of a calibrated surgical robot, the system 10 utilizes a combination of advanced kinematic analysis, real-time velocity tracking, and raycasting within the digital environment as described below. This approach enables the identification of potential collision scenarios between the robotic arms themselves, as well as between the arms and human operators or patients. The system 10 continuously analyzes the kinematics of each robotic arm 40a-d, including their positions, orientations, and velocities. By monitoring these parameters in real-time, the digital twin 100 is used to predict potential collision paths based on current trajectories and speeds. Raycasting may be employed to project virtual rays from the robotic arms in the digital twin 100 in the direction of their movement. These rays are used to detect potential collisions with other objects in their path by checking for intersections with other arms or obstacles within the digital twin environment. Collisions that may be ex vivo collisions between arms and with assist or any other object in the surgical theater as well as in vivo collision between instruments and/or with anatomy, which is detected in the digital twin 100 by interaction between digital end effector and a depth map.

For detecting potential collisions between the robotic arms and humans, the system integrates external cameras equipped with human pose estimation technologies. This allows for the precise tracking of human positions and movements, enabling the prediction of arm-to-person collisions. When a potential collision is detected, the system highlights the involved components in the digital twin 100, providing immediate visual feedback to the user.

The digital twin 100 may also provide visualization of collisions from multiple preset viewpoints. This feature allows users to quickly understand the context and specifics of the potential collision without manually adjusting the camera view. Upon detecting a potential collision, the system generates and displays recommendations for mitigation strategies. These may be rendered as wireframe corrections overlaid on the digital twin, suggesting alternative paths or movements to avoid the collision. Various audio and/or visual alerts may be emitted or displayed on the GUI 102 of varying intensity based on the likelihood and proximity of a collision.

The digital twin visualization adapts based on the proximity of the robotic arms to each other. For imminent collisions, a “stadium view” is automatically displayed as a picture-in-picture within the first display screen 32 or in a selected screen corner, providing a comprehensive overview of the situation. This view can be configured according to user preferences prior to use. The system 10 may also incorporate an adaptive arm movement control mechanism that adjusts the trajectory of the robotic arms based on impending collision assessments. This proactive adjustment slows down or alters the movement of the arms to prevent collisions.

The digital twin 100 may be temporarily deregistered or decoupled from the system 10, where one or more virtual components, e.g., arms or instrument, are controlled separately from the components of the system 10. This decoupling may be activated and deactivated by the user at any desired moment and is suitable for testing if a maneuver, e.g., to clear a collision, would cause the instrument to collide with internal anatomy. Conversely, the decoupling may be used to test if a maneuver would result in a collision.

To model and represent the access ports 55a-d and surrounding environment accurately, the system 10 may employ external cameras and sensors. These devices capture real-time data about the operational space, including the positioning of surgical instruments, the robotic arms 40a-d, and any obstacles. This information is integrated into the digital twin to enhance the collision prediction and detection capabilities, providing a comprehensive understanding of the spatial dynamics within the surgical environment.

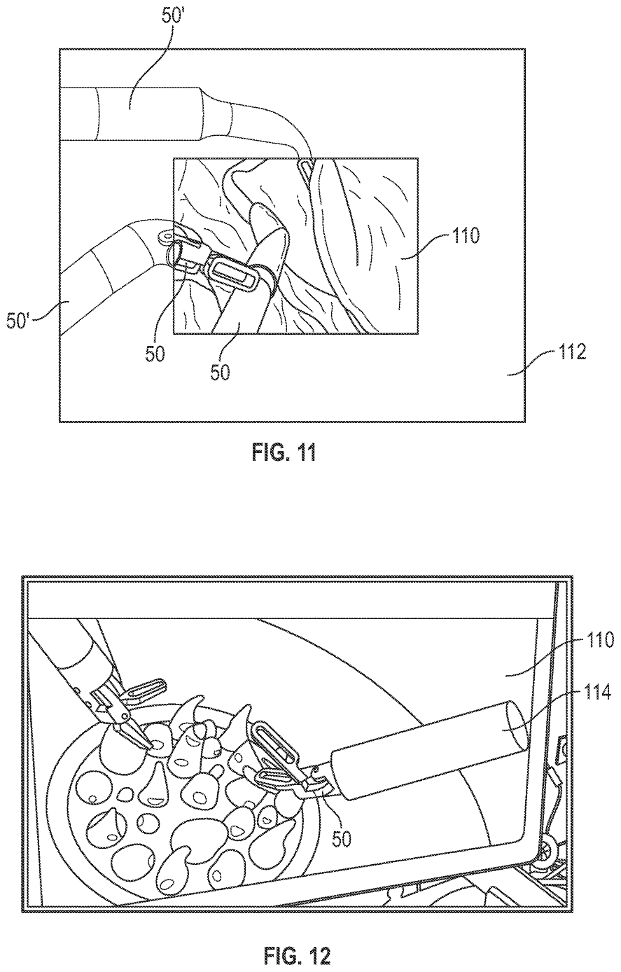

The digital twin 100 may also be used to enhance the laparoscopic view of the camera 51 displayed on the first display screen 32. With reference to FIG. 11, the laparoscopic view may be extended by a wider virtual view provided by the digital twin 100 such that the center of the display includes the laparoscopic video feed 110 provided by the camera 51 and an outer region 112 provided by the digital twin 100. The video feed from the camera 51 shows the physical instruments 50 while the virtual field of view extension shows virtual instruments 50′ extending from physical instruments 50 captured in the video feed 110. The virtual instruments 50′ provide for tool awareness of tools outside the field of the camera 51. The virtual field of view may be toggled to locate the source of instrument collisions without repositioning of the camera 51. In addition to virtual instruments 50′ provided by the digital twin 100, tissue structures and organs may be rendered on the virtual view as well based on intra of interoperative imaging data, e.g., previous camera passes. Instruments that are completely outside the field of view of the camera 51 may be indicated on the laparoscopic video feed 110 using indicators pointing in the direction of the out of view instruments.

The digital twin 100 may also include an interactive setup and reconfiguration tool that enables surgical teams to optimize the positioning of mobile carts 60a-d as shown in FIG. 5 and robotic arms 40a-d around the surgical table 90. This tool provides for both preoperative planning and intraoperative adjustments, enhancing efficiency and reducing the need for physical repositioning. The tool provides for preoperative virtual setup including virtual access port placement in the digital twin 100. Before surgery, the digital twin 100 allows for the placement of virtual access ports on a model of the patient, based on the surgical approach and patient anatomy. The placements are optimized for range of motion and accessibility. The tool also provides for cart positioning simulation. Surgeons can manipulate virtual representations of the carts 60a-d around the 3D model of the surgical table 90, ensuring that each cart is appropriately positioned for optimal access and instrument reach. Furthermore, the tool provides for robotic arm path planning. After the carts 60a-d are positioned, the digital twin 100 simulates the reach of the robotic arms, ensuring they can reach the access ports without risk of collision and within their operational range of motion. The tool may also provide a range of preset configurations based on different surgical procedures, which can be further customized for the specifics of each case.

In addition to preoperative setup, the digital twin 100 may also be used for intraoperative reconfiguration to provide for dynamic adjustments and real-time feedback. During surgery, if an obstacle is encountered or a range of motion is limited, the digital twin 100 provides a virtual environment to test different configurations of robotic arms 40a-d and instruments 50 to determine the best course of action without interrupting the surgical workflow. As changes are made to the digital twin 100, the system 10 provides immediate visual and data-driven feedback on the feasibility and safety of the proposed adjustments.

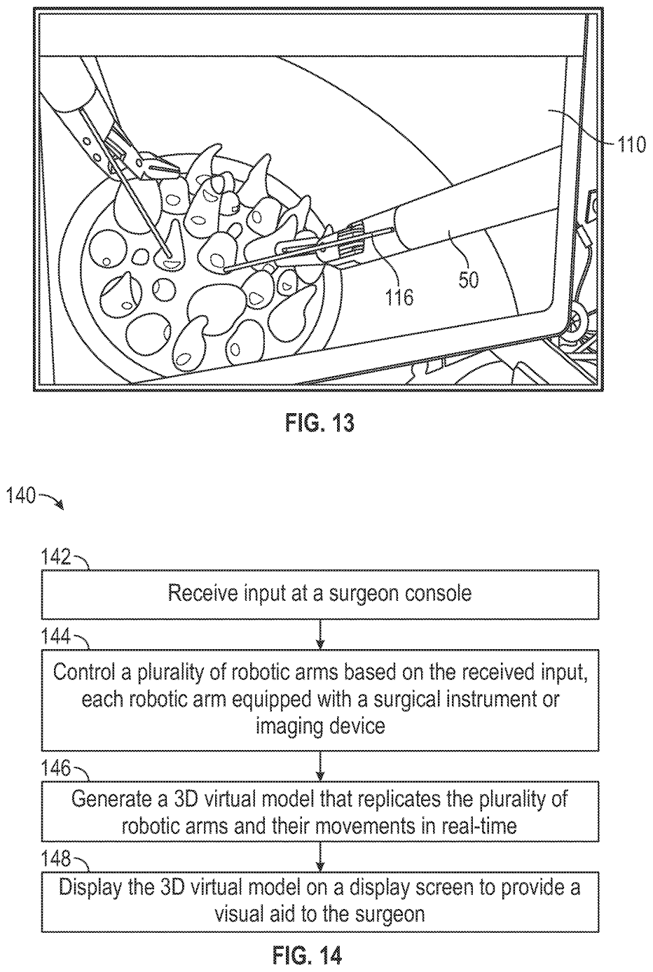

With reference to FIGS. 12 and 13, the digital twin 100 may be used to generate augmented reality (AR) overlays that are then displayed over the laparoscopic video feed 110. As shown in FIG. 12, an AR overlay 114 is provided to highlight the virtual tool shaft in different colors (e.g., green, yellow, red) or in different intensities of a single color (e.g., red) based on force feedback. This will provide users direct visual aid of how much force is being applied to each instrument/arm. The force is measured by torque sensors (not shown) in the instrument 50 or the IDU 52 and provided to the digital twin 100, which includes a virtual version of the instrument 50. The virtual portion of the instrument 50 is then used as an overlay on the actual instrument 50 shown on the first display screen 32.

Another type of AR overlay 116 is illustrated in FIG. 13, which is a line projection generated from the digital twin 100. The AR overlay 116 may be used to guide instrument insertion to target anatomy. Distance to target anatomy may also be highlighted based on a dense depth map, which may be obtained using the images from the camera 51. The dense depth map is a pixel-by-pixel representation of the 3D structure of the scene viewed by the laparoscopic camera 51. The dense depth map is generated by processing the images and comparing the differences between corresponding points in the two images. For every pixel in the left image, the system finds the matching pixel in the right image. The disparity in their positions is inversely proportional to the distance of the corresponding point in the scene from the camera; nearer objects have greater disparity. The resulting dense depth map is a grid where each value indicates the calculated distance of that point in the scene from the camera. This map is “dense” because depth is provided at every pixel, rather than for a sparse set of points, which allows for a very detailed reconstruction of the 3D structure. Such depth maps are critical in robotic surgery for tasks such as navigation, object recognition, and precise manipulation, where accurate spatial information is necessary. In embodiments, where the instrument 50 includes multiple articulated joints, multiple line extensions may be shown. More complex (e.g., curved) paths can be generated to avoid collisions or to expand workspace at the target site.

Since the digital twin 100 represents a copy of the system 10, the digital twin 100 may include its own control inputs such a secondary surgeon console (FIG. 8), another set of controllers attached to the control tower 20, or any other input means. The secondary controls are used to control the digital twin 100, which is then projected as an AR overlay. A second operator may then control the digital twin 100 to provide real time guidance to the surgeon driving the physical system 10. In addition to intraoperative visualization of manipulated instruments of the digital twin 100, accompanying instructions may be provided to illustrate use of controllers, pedals, etc. to achieve the movement as illustrated by the digital twin 100.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A surgical robotic system comprising:
a plurality of robotic arms each of which includes a surgical instrument;
a surgeon console configured to receive input to control at least one robotic arm of the plurality of robotic arms;
a controller configured to generate a 3D virtual model including a plurality of virtual arms replicating the plurality of robotic arms, wherein the 3D virtual model is moved in response to the input; and
a display screen configured to display the 3D virtual model.

2. The surgical robotic system according to claim 1, wherein the controller is further configured to detect a collision between at least two robotic arms of the plurality of robotic arms.

3. The surgical robotic system according to claim 1, wherein the controller is further configured to predict a collision between at least two robotic arms of the plurality of robotic arms.

4. The surgical robotic system according to claim 1, wherein the controller is further configured to generate a range of motion limits for the surgical instrument.

5. The surgical robotic system according to claim 4, wherein the display screen is further configured to display the range of motion limits.

6. The surgical robotic system according to claim 1, wherein the controller is further configured to deregister at least one virtual arm of the 3D virtual model from a corresponding robotic arm of the plurality of robotic arms.

7. The surgical robotic system according to claim 1, wherein the surgeon console is configured to control at least one virtual arm of the plurality of virtual arms in response to the input without controlling the at least one robotic arm of the plurality of robotic arms.

8. The surgical robotic system according to claim 1, wherein the surgeon console is configured to control at least one virtual arm in response to the input and the at least one robotic arm of the plurality of robotic arms.

9. The surgical robotic system according to claim 8, further comprising a secondary control console configured to receive input to control the at least one virtual arm of the plurality of virtual arms.

10. A surgical robotic system, comprising:
a surgeon console for input reception;
a control tower connected to a plurality of movable carts, each cart supporting a robotic arm equipped with a surgical instrument, wherein the control tower processes input from the surgeon console to control movements of the robotic arms and attached instruments;
a video processing device configured to output a processed video stream of a surgical site; and
a digital twin generator configured to create a real-time virtual representation of the robotic arms and instruments, displayed on a surgeon console display, enhancing operational control and surgical precision.

11. The system of claim 10, wherein the digital twin generator utilizes sensor data from the robotic arms to update the virtual representation in real-time.

12. The system of claim 10, wherein the surgeon console display provides a multi-view display of the surgical site and the digital twin.

13. The system of claim 10, wherein the digital twin simulates potential movements of the robotic arms and instruments.

14. The system of claim 10, further including an adaptive movement control mechanism for the robotic arms to avoid detected potential collisions.

15. A method for operating a surgical robotic system, comprising:
receiving input at a surgeon console;
controlling a plurality of robotic arms based on the received input, each robotic arm equipped with a surgical instrument;
generating a 3D virtual model that replicates the plurality of robotic arms and their movements in real-time; and
displaying the 3D virtual model on a display screen to provide a visual aid to the surgeon.

16. The method of claim 15, further comprising predicting potential collisions between the robotic arms based on their movements.

17. The method of claim 15, wherein generating the 3D virtual model includes simulating interaction forces between instruments and tissue.

18. The method of claim 15, further comprising adjusting the movements of the robotic arms to avoid predicted collisions.

19. The method of claim 15, wherein displaying the 3D virtual model includes providing visual indications of range of motion limits for the surgical instruments.

20. The method of claim 15, further comprising using the 3D virtual model to simulate surgical procedures prior to execution.
